(19) United States
(12) Patent Application Publication
(10) Pub. No.: US 2006/0270732 A1
(43) Pub. Date: Nov. 30, 2006

(54) Antioxidant supplement compostion and method of use

(75) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(73) Assignee: Suracell, Inc., Montclair, NJ (US)

(21) Appl. No.: 11/438,600

(22) Filed: May 22, 2006

Related U.S. Application Data
(60) Provisional application No. 60/685,142, filed on May 26, 2005.

(51) Int. Cl. A61K 31/385; A61K 31/355; A61K 31/198; A61K 31/05

(52) U.S. Cl. 514/440; 514/562; 514/733; 514/458

(57) ABSTRACT

An antioxidant supplement composition is provided, which contains vitamin E, resveratrol, and alpha-lipoic acid. The supplement composition can further contain N-acetyl-L-cysteine. Further provided is a method of using the antioxidant supplement composition for enhancing an individual's resistance to oxidative damages. The method includes administering the antioxidant supplement composition daily in a dosage contains from 50 to 200 IU of vitamin E, from 5 to 40 mg of resveratrol, from 50 to 400 mg of alpha-lipoic acid. In a higher strength treatment, the daily dosage further includes from 150 to 600 mg of N-acetyl-L-cysteine.

### CROSS REFERENCE TO RELATED APPLICATION

This application claims the benefit under 35 USC 119(e) of the provisional patent application Ser. No. 60/685,142, filed May 26, 2005, which is herein incorporated by reference in its entirety.

### FIELD OF THE INVENTION

The present invention relates to antioxidant supplement compositions and the method of use thereof for supplementing antioxidants to a person to enhance an individual's resistance to oxidative damages caused by free radical activities.

### BACKGROUND OF THE INVENTION

Scientific observation has shown that aging causes reduced ability to use calories from food, reduced function of hormones, depressed enzyme function, and reduced ability to fight disease. The most prevalent explanation for this has been the oxidative theory, otherwise known as the free radical theory of aging, which believes that aging results from an accumulation of changes caused by reactions in the body initiated by highly reactive molecules known as free radicals.

Free radicals promote beneficial oxidation that produces energy and kills bacterial invaders, but in excess through accumulation, they can disturb cell structure, resulting in cellular damage in protein, fat and DNA molecules. This is believed to contribute to aging, as well as various other health problems. To resist oxidative damages caused by free radical activity, scientists have been studying the effects of increasing antioxidant levels in individuals through diet and dietary supplements. It is believed that the body uses food components, such as Vitamin C, Vitamin E, and beta-carotene, as well as a variety of enzymes to destroy free radicals by beneficially combining and inactivating them and preventing cellular damage. Antioxidants donate one of their own electrons, stopping free radical chain reactions in the body.

Giampapa, The Basic Principles and Practice of Anti-Aging Medicine & Age Management, Self-published, Nespitt Granphics Inc., 2003, provides a review on aging, with a focus on understanding the oxidative stress and gene expression. More specifically, among the inflammatory chain activities triggered by free radicals, it is known that the transcription factors NF-kB and activator protein 1 (AP-1) are activated by free radicals and pro-inflammatory cytokines, which are generated by free radical activity. NF-kB and AP-1 play critical roles in the regulation of pro-inflammatory cytokines and related proteins, and collagen-digesting enzymes. Because free radicals and pro-inflammatory cytokines activate each of these transcription factors, the result is a self-reinforcing pro-inflammatory cycle. This pro-inflammatory cycle, together with the free radical damage to the cell membrane, produce intracellular free radicals that oxidize the polyunsaturated fatty acids (PUFA) rich membranes surrounding the cytoplasm's organelles, mitochondria and nucleus. It is know that the mitochondria produce cellular energy via the ATP cycle, and most repair and reproduction activities occur via production of proteins by organelles, as directed by the cell's genetic material. Thus, the pro-inflammatory cycle initiated by oxidative stress on the cell membrane gradually weakens the most basic functions of cells.

Antioxidant supplements believed to be useful in fighting age-related diseases include vitamin A and beta carotene, vitamin C, vitamin E, selenium, coenzyme Q-10, certain vitamin B complex, and alpha lipoic acid. Alpha lipoic acid is one of the most powerful antioxidants, it has been used in conjunction with vitamin C and vitamin E to assist in slowing down the aging process. Other natural substances are also known to have antioxidant properties. For example, green tea, turmeric, hawthorn, ginkgo biloba, red clover and bilberry.

Vitamin E protects cell membranes, especially in the lungs and red blood cells. It particularly protects fatty acids against oxidative damage caused by various pollutants, peroxides, and free radicals formed during metabolic processes. It aids in the prevention of lipofuscin, an oxidized fat that has been implicated in the aging process. If insufficient vitamin E is present, PUFAs may become oxidized in the body. This causes various toxins to be formed. These toxins may lead to chromosomal damage and the formation of cancers. Evidence has been presented that supplemental vitamin E along with vitamin C may greatly inhibit the formation of mutagens in the feces, thus preventing the formation of polyps and cancer. Vitamin E may protect the liver and the rest of the body against environmental pollutants such as ozone and other constituents of smog.

Resveratrol has been reported as having potent antioxidant activity and playing a significant role in the repair and maintenance of DNA strands. Resveratrol has been used recently for caloric mimic therapy.

N-acetyl-L-cysteine is an amino acid and antioxidant. N-acetyl-L-cysteine has been shown to play a protective role against a variety of toxic hazards such as cigarette smoke, auto exhaust, certain herbicides, and overdoses of acetaminophen. It has also been shown to have antiviral effects in patients with HIV due to inhibition of viral stimulation by reactive oxygen intermediates.

It is desirable to provide an improved antioxidant supplement composition including multiple antioxidants which function synergetically to enhance an individual's resistance to oxidative damages.

### SUMMARY OF THE INVENTION

In one aspect, the present invention is directed to an antioxidant supplement composition. In one embodiment, the supplement composition comprises effective amounts of vitamin E, resveratrol, and alpha-lipoic acid. In a further embodiment, the supplement composition further comprises an effective amount of N-acetyl-L-cysteine. Moreover, the supplement composition also comprises pharmaceutically acceptable excipients, and can be provided in a form for oral administration.

In a further aspect, the present invention is directed to a method of supplementing antioxidants to a person. In one embodiment, the method comprises orally administering an antioxidant supplement composition daily in a dosage comprising from about 50 IU to about 200 IU of vitamin E, from about 5 mg to about 40 mg of resveratrol, and from about 50 mg to about 400 mg of alpha-lipoic acid. In another embodiment, the method comprises orally administering an antioxidant supplement composition daily in a dosage comprising from about 50 IU to about 200 IU of vitamin E, from about 5 mg to about 40 mg of resveratrol, from about 50 mg to about 400 mg of alpha-lipoic acid, and from about 150 mg to about 600 mg of N-acetyl-L-cysteine.

### DETAILED DESCRIPTION OF THE INVENTION

In one embodiment, the present invention provides an antioxidant supplement composition for enhancing an individual's resistance to oxidative damages caused by free radical activities. The antioxidant supplement composition comprises vitamin E, resveratrol, alpha-lipoic acid and pharmaceutically acceptable excipients.

Vitamin E is a fat soluble vitamin that is composed of a group of compounds called tocopherols. Tocopherols are oily yellow liquids, water insoluble, heat and acid stable, which deteriorate with exposure to alkali, light, oxygen, and on contact with iron and lead. Tocopherol esters, which are most commonly found in foods are more resistant to extreme temperatures in food processing. There are eight tocopherols, four of them occur naturally in foods, (alpha, beta, gamma, delta or a, b, g and d). D-alpha tocopherol accounts for 80% of the activity of the vitamin. It is common that on the vitamin labels, vitamin E is labeled as mixed tocopherols; this in general indicates that 80% of the natural d-alpha tocopherol is present with the remaining 20% being a mixture of b, g and d. Vitamin E is found in cell membranes where it protects the phospholipid membrane from oxidative stress. Alpha-tocopherol, the most abundant and most active form of the vitamin E family, is the principle chain breaking antioxidant in tissue and plasma (Recommended Dietary Allowances 99-101, Nat'l Research Council, 10th ed., 1989). Vitamin E inhibits the oxidation of unsaturated fatty acids by trapping peroxyl free radicals.

In one embodiment, the supplement composition of the present invention comprises vitamin E in the form of d-alpha-tocopheryl succinate with mixed tocopherols, preferably in an amount from about 50 IU to about 200 IU in one dosage. D-alpha tocopheryl succinate is made from succinic acid, which is an ester form of d-alpha tocopherol. Succinate is an organic substance which naturally occurs in the body. In a preferred embodiment, the supplement composition comprises about 100 IU of vitamin E in the form of d-alpha-tocopheryl succinate with mixed tocopherols in one dosage.

Herein, one dosage is also referred to as one serving. If the antioxidant supplement composition is provided in the form of tablet, one dosage can be either one tablet, or two to three tablets. For example, 100 IU of vitamin E in one dosage can be provided in one tablet as shown in Supplement Composition A of Example 1, or in two tablets as shown in Supplement Composition B of Example 2. The size and number of tablets may depend on the manufacturability, which may further depend on the properties of the components and the pharmaceutically acceptable excipients used.

Resveratrol is a unique compound produced by the skins of grapes, grapevines and other plants and their roots in response to environmental stresses. Resveratrol has potent antioxidant activity and can assisting preventing free-radical damage throughout the body, providing protective support to the cardiovascular system, and supporting the repair and maintenance of DNA strands. In one embodiment, the supplement composition of the present invention comprises resveratrol, preferably in an amount from about 5 mg to about 40 mg in one dosage. In one preferred embodiment, the supplement composition comprises about 10 mg of resveratrol in one dosage. In another preferred embodiment, the supplement composition comprises about 20 mg of resveratrol in one dosage.

Alpha lipoic acid is both a lipid- and water-soluble antioxidant that works synergistically with other antioxidants in the cell's mitochondria. Alpha-lipoic acid is a cofactor for several regulatory enzymes, including pyruvate dehydrogenase, and appears to have an effect on glucose transport and utilization. Alpha-lipoic acid also increases tocopherol activity and acts as a metal chelator. In one embodiment, the supplement composition of the present invention comprises alpha-lipoic acid, preferably in an amount from about 50 mg to about 400 mg in one dosage. In one preferred embodiment, the supplement composition comprises about 100 mg of alpha-lipoic acid in one dosage. In another preferred embodiment, the supplement composition comprises about 200 mg of alpha-lipoic acid in one dosage.

In a further embodiment, the antioxidant supplement composition of the present invention further comprises N-acetyl-L-cysteine. N-acetyl-L-cysteine is an amino acid and antioxidant. N-acetyl-L-cysteine is a precursor in the body to the critical antioxidant glutathione, which is produced within cells, particularly by the liver. N-acetyl L-cysteine increases glutathione levels, protecting cells against chrysotile toxicity.

It has been found that the combination of N-acetyl-L-cysteine with the other active components described above provides a further enhanced antioxidant composition, which is particularly suitable for those individuals who are prone to oxidative damages due to generic predisposition, clinical conditions, advanced aging, or poor environmental impact. In one embodiment, the supplement composition of the present invention comprises N-acetyl-L-cysteine, preferably in an amount from about 150 mg to about 600 mg in one dosage. In a preferred embodiment, the supplement composition comprises about 300 mg of N-acetyl-L-cysteine in one dosage.

As a convenient form of dietary supplement, the supplement compositions described above are provided in the form of tablet. However, it should be understood that tablet is only one of various convenient dosage forms which can be used for the supplement composition. Other suitable forms include hard or soft-gelatin capsules, powders, or liquid dosage forms, such as elixirs, syrups, dispersed powders or granules, emulsions, or aqueous or oily suspensions. When other dosage forms are used, the amounts of the active components in one dosage remain the same, however, the concentration of the component in different pharmaceutical media can be different.

Preferably, the supplement composition is formulated as a tablet, and as such it can contain pharmaceutically acceptable excipients, according to methods and procedures well known in the art. As used herein, “excipients” means substances that are of little or no therapeutic value, but useful in the manufacture and compounding of various pharmaceutical preparations, which form the medium of the supplement composition. These substances include coloring, flavoring, and diluting agents; emulsifying and suspending agents; ointment bases; pharmaceutical solvents; antioxidants and preservatives for the product; and miscellaneous agents. Suitable excipients are described in Remington's Pharmaceutical Sciences, Mack Publishing Company, a standard reference text in this field, which is incorporated herein by reference in its entirety.

As used herein, “tablets” are solid pharmaceutical dosage forms containing active ingredients with or without suitable diluents and prepared either by compression or molding methods well known in the art. Although tablets are most frequently discoid in shape, they may also be round, oval, oblong, cylindrical, or triangular. They may differ greatly in size and weight depending on the amount of active ingredients present and the intended method of administration. They are divided into two general classes, (1) compressed tablets, and (2) molded tablets or tablet triturates. In addition to the active ingredients, tablets contain a number of inert excipients or additives. A first group of such excipients includes those materials that help to impart satisfactory compression characteristics to the formulation, including diluents, binders, and lubricants. A second group of such excipients helps to give additional desirable physical characteristics to the finished tablet, such as disintegrators, colors, flavors, and sweetening agents. Compressed tablets can be uncoated or can be sugar coated or film coated by known techniques to mask any unpleasant taste and protect the tablet from the atmosphere, or enteric coated for selective disintegration and adsorption in the gastrointestinal tract.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and other suitable materials. As used herein, “binders” are agents used to impart cohesive qualities to the powdered material. Binders insure the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and other suitable materials. As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. As used herein, “coloring agents” are chemicals that give tablets a more pleasing appearance, and in addition help the manufacturer to control the product during its preparation and help the user to identify the product. Any of the approved certified water-soluble FD&C dyes, mixtures thereof, can be used to color tablets.

Example 1 illustrates an exemplary supplement composition of the present invention, which is considered as a regular strength formula. Example 2 illustrates another exemplary supplement composition of the present invention, which is considered as an extra strength formula.

In a further aspect, the present invention provides the method of using the supplement compositions described above as a dietary supplement to individuals, particularly those in need thereof. Preferably, the supplement composition is administrated daily. The preferred daily dosage includes from about 50 to about 200 IU of vitamin E, from about 5 to about 40 mg of resveratrol, from about 50 to about 400 mg of alpha-lipoic acid. When the composition contains N-acetyl-L-cysteine, the daily dosage further includes from about 150 to about 600 mg of N-acetyl-L-cysteine.

Supplement Composition A of Example 1 is considered as a regular strength antioxidant formula for enhancing an individual's resistance to oxidative damages. The preferred daily dosage is one tablet of Supplement Composition A, which includes about 100 IU of vitamin E, about 10 mg of resveratrol, and about 100 mg of alpha-lipoic acid. Supplement Composition B of Example 2 is considered as an extra strength antioxidant formula, which is particularly suitable for individuals who are prone to oxidative damages due to genetic predisposition, clinical conditions, advanced aging, or poor environmental impact, and therefore, particularly in need of supplementing antioxidant. For these individuals, a preferred daily dosage is two tablets of Supplement Composition B, which has a daily dosage of about 100 IU of vitamin E, about 20 mg of resveratrol, about 200 mg of alpha-lipoic acid, and about 300 mg of N-acetyl-L-cysteine.

A method of determining an individual's genetic predisposition for deficiency in oxidation, in other words more prone to oxidative damages, is described in a co-pending patent application Ser. No. 60/796,423, entitled “Method of Determining Genetic Predisposition for Deficiency in Health Functions Using SNP Analysis”, which is herein incorporated by reference in its entirety. More specifically, upon performing a SNP genotyping assay of a biological sample collected from an individual, the individual's genetic predisposition for oxidation can be determined by using a specific oxidation SNP panel which comprises predetermined oxidation identifier SNPs. Such a SNP analysis determines and identifies an individual's genetic predisposition for oxidation as normal, sub-normal, and deficient. The individual whose genetic predisposition for oxidation is identified being deficient is more likely to develop clinical conditions directly or indirectly related to oxidation process.

In a further aspect, the method of the present invention provides a suitable supplement composition based on an individual's genetic predisposition for oxidation. In one embodiment, an individual whose genetic predisposition for oxidation is sub-normal is recommended to take one tablet of Supplement Composition A of Example 1 daily. In another embodiment of the present invention, an individual whose genetic predisposition for oxidation is deficient is recommended to take two tablets of Supplement Composition B of Example 2 daily. By providing a suitable antioxidant supplement composition based on an individual's genetic predisposition, the method of the present invention can more effectively assist in enhancing the individual's resistance to oxidation and reducing the likelihood of the individual in developing clinical conditions caused by oxidative damages.

The following examples are illustrative of the invention and are in no way to be interpreted as limiting the scope of the invention, as defined in the claims.

### EXAMPLE 1

A composition of the following formulation was prepared in tablet form, including the appropriate excipients, by standard methods known to those of ordinary skill in the art:

TABLE 1
Antioxidant Supplement Composition A

| | Amount Per Serving |
|---|---|
| Contents | (in one tablet) |
| d-alpha-tocopheryl succinate | 100 IU |
| and with mixed tocopherols | |
| Resveratrol | 10 mg |
| Alpha-lipoic acid | 100 mg |

Other ingredients include dicalcium phosphate, microcrystalline cellulose, croscarmellose sodium, stearic acid, magnesium stearate, silica, and pharmaceutical glaze. In this example, each tablet has a weight ranging from about 650 mg to about 800 mg.

### EXAMPLE 2

A composition of the following formulation was prepared in caplet form, including the appropriate excipients, by standard methods known to those of ordinary skill in the art:

TABLE 2
Antioxidant Supplement Composition B

| | Amount Per Serving |
|---|---|
| Contents | (in two tablets) |
| d-alpha-tocopheryl succinate | 100 IU |
| Resveratrol | 20 mg |
| Alpha-lipoic acid | 200 mg |
| N-Acetyl-L-Cysteine | 300 mg |

Other ingredients include dicalcium phosphate, microcrystalline cellulose, croscarmellose sodium, stearic acid, magnesium stearate, silica, and pharmaceutical glaze. In this example, each tablet has a weight ranging from about 650 mg to about 800 mg.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. An antioxidant supplement composition comprising effective amounts of:
(a) vitamin E;
(b) resveratrol; and
(c) alpha-lipoic acid.

2. The antioxidant supplement composition of claim 1 further comprising an effective amount of N-acetyl-L-cysteine.

3. The antioxidant supplement composition of claim 1, wherein said vitamin E is in the form of d-alpha-tocopheryl succinate with mixed tocopherols.

4. The antioxidant supplement composition of claim 3, wherein said composition comprises from about 50 IU to about 200 IU of said vitamin E in one dosage.

5. The antioxidant supplement composition of claim 1, wherein said composition comprises from about 5 mg to about 40 mg of said resveratrol in one dosage.

6. The antioxidant supplement composition of claim 1, wherein said composition comprises from about 50 mg to about 400 mg of alpha-lipoic acid in one dosage.

7. The antioxidant supplement composition of claim 2, wherein said composition comprises from about 150 mg to about 600 mg of N-acetyl-L-cysteine in one dosage.

8. The antioxidant supplement composition of claim 1 further comprising pharmaceutically acceptable excipients.

9. The antioxidant supplement composition of claim 1, wherein said composition is in a form for oral administration.

10. An antioxidant supplement composition comprising in one dosage:
(a) from about 50 IU to about 200 IU of vitamin E;
(b) from about 5 mg to about 40 mg of resveratrol; and
(c) from about 50 mg to about 400 mg of alpha-lipoic acid.

11. The antioxidant supplement composition of claim 10, wherein said composition further comprises from about 150 mg to about 600 mg of N-acetyl-L-cysteine

12. The antioxidant supplement composition of claim 10, wherein said vitamin E is in the form of d-alpha-tocopheryl succinate with mixed tocopherols including alpha, beta, delta and gamma tocopherols.

13. The antioxidant supplement composition of claim 10 further comprising pharmaceutically acceptable excipients.

14. The antioxidant supplement composition of claim 10, wherein said composition is in a form for oral administration.

15. A method for supplementing antioxidants to a person comprising administering an antioxidant supplement composition daily in a dosage comprising from about 50 IU to about 200 IU of vitamin E, from about 5 mg to about 40 mg of resveratrol, and from about 50 mg to about 400 mg of alpha-lipoic acid.

16. The method of claim 15, wherein said antioxidant supplement composition further comprises in said dosage from about 150 mg to about 600 mg of N-acetyl-L-cysteine.
